(19) United States
(12) Patent Application Publication
(10) Pub. No.: US 2015/0190431 A1
(43) Pub. Date: Jul. 9, 2015

(54) METHOD FOR ISOLATING STEM CELLS AND THEIR USE IN CELL THERAPY

(71) Applicants: INSTITUT NATIONAL DE LA RECHERCHE AGRONOMIQUE - INRA, Paris (FR); ECOLE NATIONALE VETERINAIRE, AGROALIMENTAIRE ET DE L'ALIMENTATION DE NANTES-ATLANTIQUE (ONIRIS), Nantes Cedex 3 (FR)

(72) Inventors: Karl Rouger, Orvault (FR); Yannick Cherel, Nantes (FR)

(21) Appl. No.: 14/407,843

(22) PCT Filed: Jun. 12, 2013
(86) PCT No.: PCT/EP2013/001716

§ 371 Date: Dec. 12, 2014

Related U.S. Application Data
(60) Provisional application No. 61/659,538, filed on Jun. 14, 2012.

(51) Int. Cl. A61K 35/34; C12N 5/0775

(52) U.S. Cl. CPC A61K 35/34; C12N 5/0662; C12N 2509/00

(57) ABSTRACT

The invention relates to a method for isolating muscle-derived stem cells that can be used in cell therapy, said method comprising the steps of (i) dissociating cells from at least one muscle sample, (ii) plating the cells obtained at the end of step (i) on a non-coated cell container, (iii) isolating the cells present in the supernatant of the non-coated cell container obtained at the end of step (ii), (iv) plating the cells obtained at the end of step (iii) on a coated cell container, (v) isolating the cells present in the supernatant of the coated cell container obtained at the end of step (iv), (vi) repeating, or not, the steps (iii) and (iv) at least one or two times, (vii) plating and culturing the cells isolated from the supernatant of the coated cell container obtained at the end of step (vi) until said cells have reached a confluence level of at least 50%, (viii) isolating, at the end of step (vii), the stem cells which can be used in cell therapy, wherein after expansion (a) at least 95% of said cells express CD44, CD73, (b) at least 95% of said cells express CD29, (c) at least 70% of said cells express CD90, and (d) said cells do not express CD4, CD8, CD34, CD45, CD31, CD1 17, CD144 and CD133. The invention also relates to said isolated stem cells and pharmaceutical compositions containing them.

This international patent application claims the priority of U.S. provisional application 61/659,538 filed on Jun. 14, 2012, which is herein incorporated by reference.

### FIELD OF THE INVENTION

The invention relates to a method for isolating stem cells which can be used in cell therapy, to said stem cells, to their use and compositions containing them in therapeutic strategies for pathologies or disorders that can be treated by stem cell therapy.

### BACKGROUND OF THE INVENTION

Stem cell therapy has been considered as a really interesting approach of a large number of disorders and dysfunctions. Particularly, embryonic stem cells constitute a really promising strategy but also raise an important bioethics problem.

Adult stem cell populations constitute an unlimited source of cells presenting pharmacologic and medical interest. Presence of multipotent stem cells has been reported in several tissues, including muscles. These adult stem cells have given a new impetus to cell-based therapy of several diseases, particularly among them neuromuscular diseases. More particularly, it has been demonstrated that muscle-derived stem cells contribute to injured muscle repair. Knowing that, muscle-derived stem cell therapy has been envisaged as an interesting strategy of treatment of muscular lesions and dysfunctions including dystrophies.

Duchenne muscular dystrophy is a genetic progressive muscle disease resulting from the lack of dystrophin and without effective treatment. Muscle-derived stem cells have been considered as an interesting approach for the therapy of this severe dystrophy. However, some problems still appear in the development of stem cell therapy, including difficulties to isolate and to expand stem cells of interest, and it appears to be still tricky to develop stem cell therapy making the proof of a satisfying therapeutic efficiency without major side effects such as uncontrolled risks of cancerogenesis.

Thus, there is a real need of effective treatment for dystrophies, more particularly for Duchenne Muscular Dystrophy, and new stem cell lines showing interesting properties including large expansion and proliferation capacities could represent a promising approach.

### SUMMARY OF THE INVENTION

The present invention relates to a method for isolating stem cells which can be used in cell therapy, said method comprising the steps of:
(i) dissociating cells from at least one muscle sample;
(ii) plating the cells obtained at the end of step (i) on a non-coated cell container,
(iii) isolating the cells present in the supernatant of the non-coated cell container obtained at the end of step (ii),
(iv) plating the cells obtained at the end of step (iii) on a coated cell container,
(v) isolating the cells present in the supernatant of the coated cell container obtained at the end of step (iv),
(vi) repeating, or not, the steps (iii) and (iv) at least one or two times,
(vii) plating and culturing the cells isolated from the supernatant of the coated cell container obtained at the end of step (vi) until said cells have reached a confluence level of at least 50%,
(viii) isolating, at the end of step (vii), the stem cells which can be used in cell therapy, wherein:
   a. at least 95% of said stem cells express CD44, CD73,
   b. at least 95% of said stem cells express CD29,
   c. at least 70% of said stem cells express CD90, and
   d. said stem cells do not express CD4, CD8, CD34, CD45, CD31, CD117, CD144 and CD 133.

In a particular embodiment of the invention, said method is for treating a patient suffering from a pathology that can be treated by a stem cell therapy and wherein said method comprises a further step (ix) of administering a therapeutically effective amount of the stem cells obtained at step (vi) to said patient. In a preferred embodiment, said pathology that can be treated by a stem cell therapy is Duchenne Muscular Dystrophy.

The present invention also relates to an isolated stem cell which can be obtained by the method of the invention.

Finally, the invention relates to a pharmaceutical composition comprising at least one isolated stem cell of the invention.

### DETAILED DESCRIPTION OF THE INVENTION

The inventors isolated and purified a new population of human muscle-derived stem cells, having a large expansion capacity, an ability to proliferate in suspension and a multilineage differentiation potential. These cells, that have a high amplification capacity, could be used in stem cell therapy, particularly for the treatment of a pathology or disorder affecting muscles such as dystrophies, more particularly Duchenne Muscular Dystrophy.

Thus, a first object of the invention relates to a method for isolating stem cells which can be used in cell therapy, said method comprising the steps of:
(i) dissociating cells from at least one muscle sample;
(ii) plating the cells obtained at the end of step (i) on a non-coated cell container,
(iii) isolating the cells present in the supernatant of the non-coated cell container obtained at the end of step (ii),
(iv) plating the cells obtained at the end of step (iii) on a coated cell container,
(v) isolating the cells present in the supernatant of the coated cell container obtained at the end of step (iv),
(vi) repeating, or not, the steps (iii) and (iv) at least one or two times,
(vii) plating and culturing the cells isolated from the supernatant of the coated cell container obtained at the end of step (vi) until said cells have reached a confluence level of at least 50%,
(viii) isolating, at the end of step (vii), the stem cells which can be used in cell therapy, wherein:
   a. at least 95% of said stem cells express CD44, CD73,
   b. at least 95% of said stem cells express CD29,
   c. at least 70% of said stem cells express CD90, and
   d. said stem cells do not express CD4, CD8, CD34, CD45, CD31, CD117, CD144 and CD133.

As used herein, the term “muscle sample” refers to a biological sample of muscle tissue obtained from a subject. According to the invention, the muscle tissue may be from any kind of skeletal muscle, for example Paravertebralis, Biceps femoralis, Triceps brachialis, Quadriceps muscle or Gastrocnemius muscle. Said muscle tissue is generally obtained from a biopsy but could also be provided from a necropsy done in a recent died subject. According to the invention, the muscle sample is not an embryonic sample (e.g. is not obtained from an embryo).

According to the invention, the term “subject” refers to a mammal, such as a human, but can also be another animal such as a dog, cat, a cow, a sheep, a pig, a horse, a monkey, a rat, a mouse, a rabbit, a guinea pig etc. Preferably, the subject is a human.

The step (i) consisting in the dissociation of said muscle sample obtained from a subject is necessary for the isolation of the mononucleated cells contained in said sample.

This step comprises an enzymatic dissociation that uses extracellular matrix digestive enzymes.

One skilled in the art is able to select the best enzymes and the associated concentrations so as to dissociate the muscle fibers and mononucleated cells. This choice is guided by the enzyme efficiency, wherein those skilled in the art will try to find the lowest useful enzyme concentration and the lowest incubation time for a similar efficiency. Enzymes useful in the method of the invention, alone or in association, are for example (but are not limited to) a collagenase, a trypsine, a protease, pronases, an elastase, a hyaluronidase, an actinase, a dispase.

In one embodiment of the invention, enzymes that are used for the dissociation are, alone or in association collagenases, hyaluronidases and proteases.

Particularly, the collagenase used at the step (i) of the method of the invention is a type VIII collagenase.

The term “type VIII collagenase” has its general meaning in the art and refers to a protein that in humans is encoded by the COL8A1 gene. The term may include naturally occurring type VIII collagenases and variants and modified forms thereof. The type VIII collagenase can be from any source, but typically is from a bacterium (for example from Clostridium histolyticum). Such a type VIII collagenase is commercialized by SIGMA. According to the invention, said type VIII collagenase may be used at a concentration of 2,000 collagen digestion units/g tissue for duration between 15 to 60 minutes for the enzymatic dissociation.

Particularly, the hyaluronidase used at the step (i) of the method of the invention is a type 1-S hyaluronidase.

The term “type 1-S hyaluronidase” has its general meaning in the art and refers to a protein that in humans is encoded by the HYAL1 gene. The term may include naturally occurring type 1-S hyaluronidase and variants and modified forms thereof. The type 1-S hyaluronidase can be from any source, but typically is a bovine type 1-S hyaluronidase. Such a type 1-S hyaluronidase is commercialized by SIGMA. According to the invention, said type 1-S hyaluronidase may be used at a concentration of 1,800-3,000 units/mg solid for duration between 15 to 60 minutes for the enzymatic dissociation.

Particularly, the protease(s) used at the step (i) of the method of the invention is preferably a mixture of proteases. More particularly, it may be pronase or pronase E, which is a cocktail of protease isolated from the extracellular fluid of Streptomyces griseus. Pronase E is for example commercialized by SIGMA. According to the invention, said Pronase E may be used at a concentration of 5-10 units/mg solid for duration between 30 to 60 minutes for the enzymatic dissociation.

In a particular embodiment of the invention, the enzymatic dissociation of step (i) is done in two steps by incubating said at least one sample with (a) collagenase and hyaluronidase and (b) a cocktail of proteases.

In such a particular embodiment, said hyaluronidase may be used at a concentration between 1,800 and 3,000 units/mg solid, and a concentration of 2,000 collagen digestion units/g tissue may be applied, both enzymes being used together for duration between 15 to 30 minutes. Said cocktail of proteases may be used at a concentration of 5-10 units/mg solid, for duration between 30 to 45 minutes.

In one embodiment, said dissociation of step (i) further comprises mechanical dissociation. Said mechanical dissociation is a method well known in the art and may be for example done by fine cutting into 1 mm³ pieces and/or aspiration and expulsion of the suspension through a pipette. Said mechanical dissociation may be realized before and/or after enzymatic dissociation. In a preferred embodiment of the invention, said mechanical is dissociation is realized before enzymatic dissociation and repeated after it.

The step (ii) consisting in the plating of the cells obtained at the end of step (i) on a non-coated cell container is realized in order to eliminate the fibroblasts present among the cells obtained at the end of step (i), e.g. it permits the enrichment in stem cells contained in the sample obtained at the end of step (i). In a particular embodiment of the invention, said step permits to eliminate at least 50% of the fibroblasts present among the cells obtained at the end of step (i) of the method of the invention.

Examples of non-coated cell container include, but are not limited to, uncoated tissue culture plastic flasks commercialized by CORNING, FALCON, NUNC or GREINER.

In one embodiment, the duration of said plating of step (ii) is of six hours or less, particularly five hours or less, more particularly four hours or less, preferably three hours or less, more preferably two hours or less, even more preferably about one hour.

According to the invention, the step (iii) of isolating the cells present in the supernatant of the non-coated cell container obtained at the end of step (ii) may be realized by centrifugation. According to the invention, the isolated cells are non-adherent cells.

In one embodiment of the invention, said centrifugation is done between 200×g and 400×g, preferably at about 300×g, and for a duration comprised between 5 and 20 minutes, preferably of about 10 minutes.

The step (iv) consisting in plating the cells obtained at the end of step (iii) on a coated cell container permits to eliminate the myoblasts present among the cells obtained at the end of step (iii) of the method of the invention, for enriching the sample obtained at the end of step (iii) in stem cells of interest. In a particular embodiment of the invention, said step permits to eliminate at least 50% of the myoblasts present among the cells obtained at the end of step (iii) of the method of the invention.

In one embodiment of the invention, said coated cell container may be for example (but is not limited to) a gelatin-coated cell container, a collagen-coated cell container, a laminin-coated cell container or a fibronectin-coated cell container. Such gelatin-coated cell container useful for the present invention may be gelatin-coated flasks, for example commercialized by SIGMA. Such collagen-coated cell container useful for the present invention may be collagen-coated culture plates or flasks for example commercialized by FALCON, CORNING, PERKIN ELMER or BD BIOSCIENCES. Such laminin-coated cell container useful for the present invention may be gelatin-coated flasks or plates, for example commercialized by BD BIOSCIENCES. Such fibronectin-coated cell container useful for the present invention may be collagen-coated culture plates or flasks for example commercialized by BD BIOSCIENCES.

In one embodiment, the duration of said plating of step (iv) is of at least 12 hours and at most of five days, preferably is comprised between one and three day(s).

According to the invention, the step (v) of isolating the cells present in the supernatant of the coated cell container obtained at the end of step (iv) may be realized by centrifugation. According to the invention, the isolated cells are non-adherent cells.

In one embodiment of the invention, said centrifugation is done between 200×g and 400×g, preferably at about 300×g, and for a duration comprised between 5 and 20 minutes, preferably of about 10 minutes.

According to the invention, the steps (iv) and (v) may be repeated, or not, at least one or two times, as mentioned at step (vi), for a better purification of the stem cells of interest.

In the particular embodiment of the invention wherein the steps (iv) and (v) are repeated twice, the duration of the plating of the step (iv) is for example of a day, the duration of the plating of the first repeated step (iv) is for example of three days and the duration of the plating of the second repeated step (iv) is for example of one day.

Cells obtained at the end of step (vi) are then plated and cultured at step (vii) until they reach a confluence level of at least 50%.

In a particular embodiment, the step (vii) of the method of the invention is done until said cells reach a confluence level of at least 50%, and at most 80%. Preferably, step (vii) is done until said cells reach a confluence level of about 75%.

In another particular embodiment, during the step (vii) of the method of the invention, the cells are passaged at least one, two or three times until they reach the desired confluence level. Preferably, cells are passaged at least three times.

Said culture is done in an amplification medium. An example of amplification medium that can be used is an amplification medium commercialized by MACOPHARMA and is preferably supplemented with growth factors, preferably human recombinant growth factors such as fibroblast growth factor, epidermal growth factor and fetal calf or human serum. Such growth factors are commercialized by PROMOCELL.

In a particular embodiment of the invention, fibroblast growth factor (FGF) is used at a concentration between 5 to 20 ng/mL, preferably of 10 ng/mL. In another particular embodiment, epidermal growth factor (EGF) is used at a concentration lower than 50 ng/mL, preferably lower than 25 ng/mL, more preferably of 10 ng/mL. In another particular embodiment, the amplification medium is not supplemented with stem cell factor (SCF).

In one embodiment of the invention, the duration of the step (vii) of cell culture is between two to four weeks, preferably three weeks.

The invention further comprises a step (viii) of isolating, at the end of step (vii), the stem cells which can be used in cell therapy, wherein:
a. at least 95%, preferably at least 99% of said stem cells express CD44, CD73,
b. at least 95%, preferably at least 99% of said stem cells express CD29,
c. at least 70%, preferably at least 80%, more preferably 90% of said stem cells express CD90, and
d. said stem cells do not express CD4, CD8, CD34, CD45, CD31, CD 117, CD144 and CD133.

This expression profile is obtained after expansion of said cells.

In a particular embodiment of the invention, 40 to 90%, preferably 50 to 90%, more preferably 55 to 75%, still more preferably 70% of the stem cells isolated at step (viii) also express CD49d.

In another particular embodiment of the invention, 20 to 60% of the stem cells isolated at step (viii) also express CD56.

In another particular embodiment of the invention, 40 to 80%, preferably 40 to 70% more preferably 45 to 65%, still more preferably 60% of the stem cells isolated at step (viii) also express CD146.

According to the invention, the expression “do not express a marker” means that less than 5%, preferably less than 3%, more preferably less than 1% of the cells express said marker.

Characterization of cells or determination of the phenotype of cells generally consists in analysis of cell markers, done by several methods well known in the art.

As used herein, the term “cellular marker” refers to any cell antigen permitting to have information (alone or in combination with other) on the cell type. Methods for characterizing a cellular antigen are well known in the art, such as flow cytometry, Western-Blot or immunocytochemistry. Anyone of these known methods may be used.

According to the invention, cell markers that could be analyzed for the identification of cell types may be specifically chosen among the following group: CD4, CD8, CD29, CD31, CD34, CD44, CD45, CD49d, CD56, CD73, CD90, CD117, CD133, CD144 and CD146.

The term “CD4” (cluster of differentiation 4) has its general meaning in the art and refers to a well known glycoprotein particularly expressed at the surface of immune cells such as T helper cells, dendritic cells, monocytes and macrophages. The term may include naturally occurring CD4 protein and variants and modified forms thereof. The CD4 protein can be from any source, but typically is a mammalian (e.g., human and non-human primate) CD4 protein, particularly a human CD4 protein. An exemplary human native CD4 protein amino acid sequence is provided in NP_{—}000607.1 and an exemplary human native CD4 protein nucleic acid sequence is provided in NM_{—}000616.4. The presence of the CD4 protein on cell surface may be assessed by classical methods well known in the art such as flow cytometry using an antibody recognizing CD4. Examples of antibodies recognizing CD4 are commercialized by BD BIOSCIENCES, CLINISCIENCES AND SEROTEC.

The term “CD8” (cluster of differentiation 8) has its general meaning in the art and refers to a well known transmembrane glycoprotein that particularly serves as a co-receptor for the T cell receptor. The term may include naturally occurring CD8 protein and variants and modified forms thereof. The CD8 protein can be from any source, but typically is a mammalian (e.g., human and non-human primate) CD8 protein, particularly a human CD8 protein. An exemplary human native CD8 protein amino acid sequence is provided in P01732 (UniProt Database) and an exemplary human native CD8 protein nucleic acid sequence is provided in NM_{—}001768. The presence of the CD8 protein on cell surface may be assessed by classical methods well known in the art such as flow cytometry using an antibody recognizing CD8. Examples of antibodies recognizing CD8 are commercialized by BD BIOSCIENCES, CLINISCIENCES AND SEROTEC.

The term “CD29” (Cluster of Differentiation 29) has its general meaning in the art and refers to a protein also called Integrin beta-1 encoded in human by the ITGB1 gene. The term may include naturally occurring CD29 protein and variants and modified forms thereof. The CD29 protein can be from any source, but typically is a mammalian (e.g., human and non-human primate) CD29 protein, particularly a human CD29 protein. An exemplary human native CD29 protein amino acid sequence is provided in NP_{—}002202.2 and an exemplary human native CD29 protein nucleic acid sequence is provided in NM_{—}002211.3. The presence of the CD29 protein on cell surface may be assessed by classical methods well known in the art such as flow cytometry using an antibody recognizing CD29. Examples of antibodies recognizing CD29 are commercialized by BD BIOSCIENCES, CLINISCIENCES, SEROTEC.

The term “CD31” (cluster of differentiation 31), also called Platelet endothelial cell adhesion molecule (PECAM-1), has its general meaning in the art and refers to a protein encoded by the PECAM1 gene in human. The term may include naturally occurring CD31 protein and variants and modified forms thereof. The CD31 protein can be from any source, but typically is a mammalian (e.g., human and non-human primate) CD31 protein, particularly a human CD31 protein. An exemplary human native CD31 protein amino acid sequence is provided in NP_{—}000433.4 and an exemplary human native CD31 protein nucleic acid sequence is provided in NM_{—}000442.4. The presence of the CD31 protein on cell surface may be assessed by classical methods well known in the art such as flow cytometry using an antibody recognizing CD31. Examples of antibodies recognizing CD31 are commercialized by BD BIOSCIENCES, CLINISCIENCES AND SEROTEC.

The term “CD34” (cluster of differentiation 34) has its general meaning in the art and refers to a cell surface glycoprotein functioning as a cell-cell adhesion factor. The term may include naturally occurring CD34 protein and variants and modified forms thereof. The CD34 protein can be from any source, but typically is a mammalian (e.g., human and non-human primate) CD34 protein, particularly a human CD34 protein. An exemplary human native CD34 protein amino acid sequence is provided in NP_{—}001020280.1 and an exemplary human native CD34 protein nucleic acid sequence is provided in NM_{—}001025109.1. The presence of the CD34 protein on cell surface may be assessed by classical methods well known in the art such as flow cytometry using an antibody recognizing CD34. Examples of antibodies recognizing CD34 are commercialized by BD BIOSCIENCES, CLINISCIENCES AND SEROTEC.

The term “CD44” (cluster of differentiation 44) has its general meaning in the art and refers to cell-surface glycoprotein involved in cell-cell interactions, cell adhesion and migration. The term may include naturally occurring CD44 protein and variants and modified forms thereof. The CD44 protein can be from any source, but typically is a mammalian (e.g., human and non-human primate) CD44 protein, particularly a human CD44 protein. An exemplary human native CD44 protein amino acid sequence is provided in NP_{—}000601.3 and an exemplary human native CD44 protein nucleic acid sequence is provided in NM_{—}000610.3. The presence of the CD44 protein on cell surface may be assessed by classical methods well known in the art such as flow cytometry using an antibody recognizing CD44. Examples of antibodies recognizing CD44 are commercialized by BD BIOSCIENCES, CLINISCIENCES AND SEROTEC.

The term “CD45” (cluster of differentiation 45), also called Protein tyrosine phosphatase receptor type C (PTPRC), has its general meaning in the art and refers to an enzyme that, in human, is encoded by the PTPRC gene. The term may include naturally occurring CD45 protein and variants and modified forms thereof. The CD45 protein can be from any source, but typically is a mammalian (e.g., human and non-human primate) CD45 protein, particularly a human CD45 protein. An exemplary human native CD45 protein amino acid sequence is provided in NP_{—}002829.2 and an exemplary human native CD45 protein nucleic acid sequence is provided in NM_{—}002838.3. The presence of the CD45 protein on cell surface may be assessed by classical methods well known in the art such as flow cytometry using an antibody recognizing CD45. Examples of antibodies recognizing CD45 are commercialized by BD BIOSCIENCES, CLINISCIENCES AND SEROTEC.

The term “CD49d” has its general meaning in the art and refers to the integrin alpha 4 subunit. The term may include naturally occurring CD49d protein and variants and modified forms thereof. The CD49d protein can be from any source, but typically is a mammalian (e.g., human and non-human primate) CD49d protein, particularly a human CD49d protein. An exemplary human native CD49d protein amino acid sequence is provided in NP_{—}000876.3 and an exemplary human native CD49d protein nucleic acid sequence is provided in NM_{—}000885.4. The presence of the CD49d protein on cell surface may be assessed by classical methods well known in the art such as flow cytometry using an antibody recognizing CD49d. Examples of antibodies recognizing CD49d are commercialized by BD BIOSCIENCES, CLINISCIENCES, SEROTEC.

The term “CD56” (cluster of differentiation 56) has its general meaning in the art and refers to a protein also called NCAM (Neutral Cell Adhesion Molecule, or NCAM1). The term may include naturally occurring CD56 protein and variants and modified forms thereof. The CD56 protein can be from any source, but typically is a mammalian (e.g., human and non-human primate) CD56 protein, particularly a human CD56 protein. An exemplary human native CD56 protein amino acid sequence is provided in NP_{—}000606.3 and an exemplary human native CD56 protein nucleic acid sequence is provided in NM_{—}000615.6. The presence of the CD56 protein on cell surface may be assessed by classical methods well known in the art such as flow cytometry using an antibody recognizing CD56. Examples of antibodies recognizing CD56 are commercialized by BD BIOSCIENCES, CLINISCIENCES, SEROTEC.

The term “CD73” (cluster of differentiation 73), also known as ecto-5′-nucleotidase or 5′-nucleotidase (5′-NT), has its general meaning in the art and refers to an enzyme that in humans is encoded by the NT5E gene. The term may include naturally occurring CD73 protein and variants and modified forms thereof. The CD73 protein can be from any source, but typically is a mammalian (e.g., human and non-human primate) CD73 protein, particularly a human CD73 protein. An exemplary human native CD73 protein amino acid sequence is provided in NP_{—}001191742.1 and an exemplary human native CD73 protein nucleic acid sequence is provided in NM_{—}001204813.1. The presence of the CD73 protein on cell surface may be assessed by classical methods well known in the art such as flow cytometry using an antibody recognizing CD73. Examples of antibodies recognizing CD73 are commercialized by BD BIOSCIENCES, CLINISCIENCES AND SEROTEC.

The term “CD90” (Cluster of Differentiation 90) or “Thy-1” has its general meaning in the art and refers to a protein commonly used as a marker of stem cells and encoded in human by the THY-1 gene. The term may include naturally occurring CD90 protein and variants and modified forms thereof. The CD90 protein can be from any source, but typically is a mammalian (e.g., human and non-human primate) CD90 protein, particularly a human CD90 protein. An exemplary human native CD90 protein amino acid sequence is provided in NP_{—}006279.2 and an exemplary human native CD90 protein nucleic acid sequence is provided in NM_{—}006288.3. The presence of the CD90 protein on cell surface may be assessed by classical methods well known in the art such as flow cytometry an antibody recognizing CD90. Examples of antibodies recognizing CD90 are commercialized by BD BIOSCIENCES, CLINISCIENCES, SEROTEC.

The term “CD117” (Cluster of Differentiation 117), also called Mast/stem cell growth factor receptor (SCFR), proto-oncogene c-Kit or tyrosine-protein kinase Kit, has its general meaning in the art and refers to a protein that in humans is encoded by the KIT gene. The term may include naturally occurring CD 117 protein and variants and modified forms thereof. The CD 117 protein can be from any source, but typically is a mammalian (e.g., human and non-human primate) CD 117 protein, particularly a human CD117 protein. An exemplary human native CD117 protein amino acid sequence is provided in NP_{—}000213.1 and an exemplary human native CD117 protein nucleic acid sequence is provided in NM_{—}000222.2. The presence of the CD117 protein on cell surface may be assessed by classical methods well known in the art such as flow cytometry using an antibody recognizing CD117. Examples of antibodies recognizing CD117 are commercialized by BD BIOSCIENCES, CLINISCIENCES AND SEROTEC.

The term “CD133” (Cluster of Differentiation 133), also called AC133 or Prominin 1 (PROM1) in human and rodents, has its general meaning in the art and refers to a member of pentaspan transmembrane glycoproteins (5-transmembrane, 5-TM). The term may include naturally occurring CD133 protein and variants and modified forms thereof. The CD133 protein can be from any source, but typically is a mammalian (e.g., human and non-human primate) CD133 protein, particularly a human CD133 protein. An exemplary human native CD133 protein amino acid sequence is provided in NP_{—}001139319.1 and an exemplary human native CD133 protein nucleic acid sequence is provided in NM_{—}001145847.1. The presence of the CD133 protein on cell surface may be assessed by classical methods well known in the art such as flow cytometry using an antibody recognizing CD133. Examples of antibodies recognizing CD133 are commercialized by MILTENYIBIOTEC.

The term “CD144” (Cluster of Differentiation 133), also Cadherin 5, type 2 or VE-cadherin (vascular endothelial), has its general meaning in the art and refers to a type of cadherin encoded by the human gene CDH5. The term may include naturally occurring CD144 protein and variants and modified forms thereof. The CD144 protein can be from any source, but typically is a mammalian (e.g., human and non-human primate) CD144 protein, particularly a human CD144 protein. An exemplary human native CD144 protein amino acid sequence is provided in NP_{—}001786.2 and an exemplary human native CD144 protein nucleic acid sequence is provided in NM_{—}001114117.1. The presence of the CD144 protein on cell surface may be assessed by classical methods well known in the art such as flow cytometry using an antibody recognizing CD 144. Examples of antibodies recognizing CD144 are commercialized by BD BIOSCIENCES, CLINISCIENCES AND SEROTEC.

The term “CD146” (Cluster of Differentiation 146), also called Melanoma Cell Adhesion Molecule (MCAM) or cell surface glycoprotein MUC18 has its general meaning in the art and refers to a 113 kDa cell adhesion molecule encoded in human by the MCAM gene. The term may include naturally occurring CD 146 protein and variants and modified forms thereof. The CD146 protein can be from any source, but typically is a mammalian (e.g., human and non-human primate) CD146 protein, particularly a human CD146 protein. An exemplary human native CD146 protein amino acid sequence is provided in NP_{—}006491.2 and an exemplary human native CD 146 protein nucleic acid sequence is provided in (mRNA) NM_{—}006500.2. The presence of the CD146 protein on cell surface may be assessed by classical methods well known in the art such as flow cytometry using an antibody recognizing CD146. Examples of antibodies recognizing CD146 are commercialized by BD BIOSCIENCES, CLINISCIENCES, SEROTEC.

In a particular embodiment, the method of the invention for isolating stem cells which can be used in cell therapy is a method for treating a patient suffering from a pathology or a disorder that can be treated by a stem cell therapy. In such an embodiment, said method comprises a further step (ix) of administering a therapeutically effective amount of said stem cells to said patient.

The term “treating” or “treatment” refers to reversing, alleviating, inhibiting the progress of, or preventing the disorder or condition to which such term applies, or one or more symptoms of such disorder or condition.

According to the present invention, a “therapeutically effective amount” of a composition or compound is one which is sufficient to achieve a desired biological effect. It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the preferred dosage can be tailored to the individual subject, as is understood and determinable by one of skill in the art, without undue experimentation.

As used herein, the term “patient” refers to a human or another mammal (e.g., primate, dog, cat, goat, horse, pig, mouse, rat, rabbit, and the like), that can be afflicted with a pathology that can be treated by a stem cell therapy. Preferably, the patient is a human.

In a particular embodiment of the invention, when the method of the invention is used for treating a patient for said pathology or disorder, the muscle sample of step (i) of the method of the invention may be obtained from (a) a subject free of muscular pathology or from (b) said patient suffering from a pathology that can be treated by a stem cell therapy. Such a muscle sample may be called “allogenic muscle sample” in case (a) or “autologous muscle sample” in case (b).

In one embodiment, the method for treating a patient suffering from a pathology or a disorder that can be treated by said therapy is applied to a patient suffering from a muscle pathology or disorder, comprising muscular lesion, injury or dysfunction. According to the invention, pathologies that can be treated by a stem cell therapy include, but are not limited to lesions, injuries or dysfunctions affecting muscular or cardiac tissues.

Muscular injuries comprise a large percentage of recreational and competitive athletic injuries, resulting from direct and indirect trauma. Muscular lesions associated with aging are also considered.

Muscular dysfunctions include muscular dystrophies such as Becker and Duchenne dystrophies, myotonic dystrophy (also known as Steinert's disease), limb-girdle muscular dystrophy, facioscapulohumeral muscular dystrophy, congenital muscular dystrophy, oculopharyngeal muscular dystrophy, distal muscular dystrophy and Emery-Dreifuss muscular dystrophy. Now, muscular dysfunction may include others myopathies.

In a preferred embodiment of the invention, said patient suffers from Duchenne Muscular Dystrophy.

Furthermore, the stem cells of the invention are able to differentiate towards different pathways. Indeed, they are not only able to follow the myogenic pathway, but also adipogenic and osteogenic pathway.

Thus, in another embodiment, the method for treating a patient suffering from a pathology or a disorder that can be treated by said therapy is applied to a patient suffering from a pathology, lesion or injury affecting osseous, cartilaginous or adipose tissue.

In a particular embodiment, said patient suffers from bone or cartilage lesion, injury or dysfunction including, but not limited to, segmental bone fractures, defects, weakness, non-unions and any type of bone augmentation. In another particular embodiment, said patient suffers from a lipodystrophy associated with an anti-HIV treatment.

Administration of selected cells may be done by injection into a given tissue or site of injury of a therapeutically effective amount of cells in solution or suspension, preferably about 10⁵ to 10⁶ cells per cm³ of tissue to be treated, in a physiologically acceptable medium. Administration of a therapeutically effective amount of said cells may also be realized by systemic delivery. Then, about 10² to 10³ cells per cm³ of tissue to be treated may be injected.

In the particular embodiment of a Duchenne Muscular Dystrophy therapy, cells isolated by the method of the invention are administered by systemic delivery (e.g. intra-arterial injection), locoregional delivery (e.g., member or part of member isolated from the general circulation during injection time), or by intramuscular injection.

The term “physiologically acceptable medium” refers to a medium that is not toxic and that is suitable for systemic administration or local injection. Physiologically acceptable media are generally adapted to the nature of a medium on which a composition is to be applied, and also to a form in which a composition is packaged.

The dose of cells administered to the patient (by any kind of administration pathway) can be repeated, depending on the patient's condition and evolution, at time intervals of days, weeks or months that have to be established by the specialist in each case.

A second object of the invention relates to an isolated stem cell which can be obtained by the method of the invention.

A third object of the invention relates to a pharmaceutical composition comprising at least one isolated stem cell of the invention.

The composition of the invention may include, in addition to the stem cell(s) of the invention, non-cellular components. Examples of such non-cellular components include but are not limited to cell culture media, which may comprise one or more of proteins, amino acids, nucleic acids, nucleotides, co-enzyme, anti-oxidants and metals. Said composition may contain a pharmaceutically acceptable carrier or excipient.

As used herein, the expression “pharmaceutically acceptable carrier” means a material, composition or vehicle, such as a liquid or solid filler, diluent, excipient, or solvent encapsulating material, involved in carrying or transporting the stem cells of the invention. Each carrier must be “pharmaceutically acceptable” in the sense of being suitable for use in contact with the tissues of patient without excessive toxicity, irritation, allergic response, or other problem or complication, commensurate with a reasonable benefit/risk ratio. Examples of such carriers are well known in the art, and may include: sugars, such as lactose, glucose buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

A composition of the invention may be provided under sterile conditions, and may be free of viruses, bacteria and other pathogens.

In one embodiment of the invention, said pharmaceutical composition comprising at least one stem cell of the invention is for the treatment of a pathology or a disorder that can be treated by stem cell therapy including lesions, injuries or dysfunctions affecting muscular or cardiac tissues, as described above.

In a preferred embodiment of the invention, said pharmaceutical composition is for the treatment of Duchenne Muscular Dystrophy.

In another embodiment, said composition may also be used for the treatment of a pathology affecting osseous, cartilaginous or adipose tissue as described above.

### EXAMPLES

Isolation and In Vitro Expansion of Human MuStem Cells.

Skeletal muscle tissues were obtained from the Paravertebralis muscles of 9 to 15-year-old patients free of known muscular pathologies. Freshly isolated skeletal muscle tissues were placed on ice in a rinsing and hypothermic preservation solution for grafts (MACOPHARMA; Tourcoing, France) containing 2% penicillin/streptomycin/fungizon (PSF, SIGMA; St. Louis, Mo., USA) and transferred to the laboratory. They were finely minced into 1 mm³ pieces using forceps and scalpel, rinsed three times in phosphate buffered saline without Ca²⁺/Mg²⁺ (PBS, PAA; Les Rumeaux, France)/2% PSF to remove fat and red blood cells and enzymatically digested for 15 min at 37° C. by using a mixture of collagenase type-VIII (2,000 U/g of tissue, Sigma) and 0.2% hyaluronidase type-1S (SIGMA) in HAM F12 medium (INVITROGEN; Saint Aubin, France) in a shaking water bath. The pre-digested tissue was centrifuged at 100×g for 5 min, the supernatant collected and neutralized with 20% (v/v) fetal calf serum (FCS; SIGMA) and placed in pre-warmed HAM F12 medium. The pellet was submitted to a second enzymatic digestion for 30 min at 37° C. with 0.125% Pronase E (SIGMA) in HAM F12/17% FCS/2% PSF in a shaking water bath. The mixture was centrifuged at 100×g for 5 min, the supernatant collected, pooled with those obtain after the first enzymatic digestion and mechanically dissociated. It was submitted to successive centrifugation (300×g, 15 min) and sequential filtering through 100-, 70-, and 40-μm pore-diameter nylon mesh (BD BIOSCIENCES; San Jose, Calif.) to obtain a single cell suspension by separation of the muscle-derived cells (MDCs) from the debris. Cells were resuspended in PBS/2% FCS/1% PSF (PBS+) and viability was assessed using 0.1% trypan blue staining (VWR; Strasbourg, France).

Human MuStem cells were isolated using a modified version of the technique described previously in canine species (Rouger et al., 2011). Freshly extracted MDCs were plated to uncoated tissue culture plastic flasks at 1.5 to 2.10⁵ viable cells/cm² in a growth medium (84% HAM F12/15% FCS/1% PSF) considering 0.15 mL/cm² for one hour. Floating cells found in the supernatant were then centrifuged (300×g, 10 min), resuspended in PBS+, counted via Mallasez chamber, and transferred to 0.1% gelatin-coated flasks (SIGMA) at 1.10⁵ viable cells/cm², whilst adherent cells that were highly enriched in fibroblasts were discarded. After 24 hours, the procedure was repeated: Floating cells were plated at 5.10⁴ viable cells/cm² on new gelatin-coated flasks in a growth medium (0.15 mL/cm²) and were kept at 37° C. in a 5% CO2 atmosphere for three days. At day 4, floating cells were plated at 2.10⁴ viable cells/cm² on new gelatin-coated flasks in a growth medium (0.15 mL/cm²). After 24 hours, floating cells were transferred to coated flasks at 5.10³ to 1.10⁴ viable cells/cm² in growth medium (0.15 mL/cm²) and maintained for another three days without medium change. Finally, the medium was eliminated by gentle pipetting and the latest poorly adherent cells (corresponding to the MuStem cells) were expanded in clinical grade medium (MACOPHARMA) supplemented to 10% FCS, 1% PSF and 10 ng/mL human recombinant basic fibroblast growth factor and 10 ng/mL human recombinant epidermal growth factor (all from PROMOCELL; Heidelberg, Germany). Once cell colony derived-cultures reached 75% confluence, cells were passaged by treatment with accutase (SIGMA) diluted 1:3 in 1×PBS for 5-6 min at 37° C., neutralized by 5% FCS, centrifuged, resuspended in PBS+, and tested for their viability using trypan blue exclusion test. Finally, they were plated at 8.10³ viable cells/cm², grown under standard condition (5% CO₂, 37° C.) and expansion medium was replaced every three days. Cells were passaged when cultures reached a confluence of 75%. Then, cells were frozen and stored in liquid nitrogen.

Characterization of Human MuStem Cells.

In vitro investigations revealed that MuStem cells are clonogenic and display a large proliferation ability (with at least 15 population doubling levels).

We performed flow cytometry for large panel of lineage markers allowing us to determine that MuStem cells exhibit a high (at least 70% of positive cells) expression for CD29, CD44, CD73 and CD90, four markers currently used to qualify mesenchymal stem cells. An expression for perivascular cell markers NG2, PDGF-Rb and CD49b is also observed with a same range. MuStem cells are negative (e.g., less than 3% of positive cells) to blood cell markers CD3, CD4, CD8, CD11a, CD11b, CD11c and CD14. Also, they do not express endothelial cell markers CD31, CD144 and VEGF-R1/R2 as well as hematopoietic cell markers CD34, CD45 and the CD133. A moderate (between 20 and 50% of positive cells) expression for the myoblast and satellite cell marker CD56 is defined. In addition to this analysis, a flow cytometry exploration of cell markers related to immunity revealed that whether CD18, CD19, CD33, CD38, CD40, CD72, CD80, CD86 and CD220 are negative, CD26, CD47, CD58, CD59 and HLA-A,B,C are expressed by at least 80% of MuStem cells. Using immunocytochemistry, we revealed a HO1-1 expression on all MuStem cell samples and that at least 80% of cells are positive to iNOS. Using Reverse Transcription Polymerase Chain Reaction (RT-PCR), we showed IL6 and IL8 expression while IL10 is missing. Moreover, we determined that MuStem cells exhibit expression of stem cell markers Oct3/4 and Klf-4 and sometimes Nanog. RT-PCR results indicated that MuStem cells are committed in myogenic lineage with an expression of the progenitor cell marker Myf5. By developing in vitro differentiation assay, we established that MuStem cells are capable to generate cells differentiating into myotubes with demonstration of multinucleated cell expressing sarcomeric isoform of myosin heavy chain and into other mesodermal cell types in lineage specific induction media such as osteocytes and/or adipocytes. We performed cell transplantation of MuStem cell population to injured Tibialis cranialis muscle of SCID mice. Four weeks after transplantation of 3.10⁵ cells, we determined by immunolabelling that MuStem cells or their progeny are able to persist into injured skeletal muscle and exhibit different tissue localization in a same way it has been demonstrated for the canine

MuStem cells after intra-muscular injection in dystrophic dogs. Indeed, cells could be observed in the cytoplasm of muscle fibers with a central or peripheral position, and in the interstitial tissue.

Administration of MuStem Cells to a Patient for Duchenne Muscular Dystrophy Therapy.

The presence of mycoplasma and bacteria in the cell culture is tested during the time of MuStem cell expansion. For transplantation, cells are detached from the culture flasks with accutase, washed and re-suspended in 0.9% NaCl solution/2.5% homologous serum. For intramuscular administration, the region to be injected is shaved, cleaned, and disinfected. Local anesthesia is done. Injections of 3×10⁶ viable MuStem cells in 300 μL are performed at a rate of 60-80 μL/min using a 100 μL Hamilton syringe with a 26-gauge needle. For systemic or locoregional delivery, MuStem cells are suspended at 2×10⁶ cells/mL in 0.9% NaCl solution/2.5% homologous serum/50 U/mL heparin. Systemic delivery is performed according to current surgery practice.

We claim:

1. A method for isolating stem cells which can be used in cell therapy, said method comprising the steps of:
(i) dissociating cells from at least one muscle sample;
(ii) plating the cells obtained at the end of step (i) on a non-coated cell container,
(iii) isolating the cells present in the supernatant of the non-coated cell container obtained at the end of step (ii),
(iv) plating the cells obtained at the end of step (iii) on a coated cell container,
(v) isolating the cells present in the supernatant of the coated cell container obtained at the end of step (iv),
(vi) repeating, or not, the steps (iii) and (iv) at least one or two times,
(vii) plating and culturing the cells isolated from the supernatant of the coated cell container obtained at the end of step (vi) until said cells have reached a confluence level of at least 50%,
(viii) isolating, at the end of step (vii), the stem cells which can be used in cell therapy, wherein:
a. at least 95% of said stem cells express CD44, CD73,
b. at least 95% of said stem cells express CD29,
c. at least 70% of said stem cells express CD90, and
d. said stem cells do not express CD4, CD8, CD34, CD45, CD31, CD 117, CD144 and CD133.

2. The method of claim 1, wherein the muscle sample of step (i) is obtained from a human subject.

3. The method of claim 1, wherein the dissociation of step (i) comprises an enzymatic dissociation.

4. The method of claim 3, wherein said enzymatic dissociation is done in two steps by incubating said at least one muscle sample with (a) collagenase and hyaluronidase and (b) a cocktail of proteases.

5. The method of claim 3, wherein the step (i) further comprises a mechanical dissociation.

6. The method of claim 1, wherein the plating of step (iv) is done on a gelatin-coated cell container or on a collagen-coated cell container.

7. The method of claim 1, wherein the step (vii) is done until said cells have reached a confluence level of 75%.

8. The method of claim 1, wherein said method is for treating a patient suffering from a pathology that can be treated by a stem cell therapy and wherein said method comprises a further step (ix) of administering a therapeutically effective amount of the stem cells obtained at step (vi) to said patient.

9. The method of claim 8, wherein said patient is a human.

10. The method of claim 8, wherein said pathology that can be treated by a stem cell therapy is (a) a muscular dystrophy chosen among the group comprising Becker and Duchenne muscular dystrophies, myotonic dystrophy, limb-girdle muscular dystrophy, facioscapulohumeral muscular dystrophy, congenital muscular dystrophy, oculopharyngeal muscular dystrophy, distal muscular dystrophy and Emery-Dreifuss muscular dystrophy, (b) a muscle lesion or (c) a muscle modification associating with aging.

11. The method of claim 8, wherein said pathology that can be treated by a stem cell therapy is Duchenne Muscular Dystrophy.

12. An isolated stem cell which can be obtained by the method of claim 1.

13. A pharmaceutical composition comprising at least one isolated stem cell of claim 12.

14. The pharmaceutical composition of claim 13 for the treatment of a pathology that can be treated by a stem cell therapy, said pathology being is (a) a muscular dystrophy chosen among the group comprising Becker and Duchenne muscular dystrophies, myotonic dystrophy, limb-girdle muscular dystrophy, facioscapulohumeral muscular dystrophy, congenital muscular dystrophy, oculopharyngeal muscular dystrophy, distal muscular dystrophy and Emery-Dreifuss muscular dystrophy, (b) a muscle lesion or (c) a muscle modification associating with aging,

15. The pharmaceutical composition of claim 13 for the treatment of Duchenne Muscular Dystrophy.
